(19) United States
(12) Patent Application Publication
(10) Pub. No.: US 2021/0046004 A1
(43) Pub. Date: Feb. 18, 2021

(54) EFFERVESCENT POWDER DELIVERY SYSTEM

(71) Applicant: Tabbrands Inc., Laguna, CA (US)

(72) Inventor: Michael Gulyas, Laguna Beach, CA (US)

(73) Assignee: TabBrands Inc.

(21) Appl. No.: 17/074,916

(22) Filed: Oct. 20, 2020

Related U.S. Application Data
(63) Continuation of application No. 16/630,647, filed on Jan. 13, 2020.
(60) Provisional application No. 62/532,142, filed on Jul. 13, 2017.

(51) Int. Cl. A61K 9/00; A23L 33/17; A23L 33/105; A61J 1/03; A61K 9/20; A61K 9/28; A61K 31/352

(52) U.S. Cl. CPC A61K 9/0095; A23L 33/17; A23L 33/105; A61K 31/352; A61K 9/2063; A61K 9/282; A61J 1/035

(57) ABSTRACT

An effervescent powder product containing cannabinoids, terpenes and cannabidiol (CBD) and a process for producing an effervescent supplement powder. The effervescent powder having a density that enables the effervescent powder to break apart and/or dissolve when it is added to or submerged in any liquid, including non-carbonated and carbonated liquids, with or without stirring. The floating delivery system yields an effervescent powder bulk density that is lower than the gastric fluid and allows the active components to remain buoyant for extended time periods without affecting the rate of gastric emptying.

### CROSS-REFERENCE TO RELATED APPLICATIONS

This application is a continuation-in-part of U.S. patent application Ser. No. 16/630,647, filed on Jan. 13, 2020 which claims priority to PCT Application No. PCTUS2018042180, filed on Jul. 13, 2018 which claims the benefit of U.S. Provisional Patent Application 62/532,142, filed on Jul. 13, 2017, which is hereby incorporated by reference in its entirety.

### TECHNICAL FIELD

The present disclosure relates generally to a delivery system, and more particularly to an effervescent product containing active ingredients of hemp or cannabis extract.

### BACKGROUND

There is a growing need for faster and more efficient mechanisms to distribute vitamins, supplements, and pharmaceutical substances to the average person in their daily life.

One example of an industry where distribution and transport is particularly troublesome is in the supplement industry, which serves a vast number of people from body-builders to vegans/vegetarians. Similar problems are faced within markets associated with trade in pharmaceuticals and additives. People consume a wide variety of supplements and additives for a variety of reasons, and these industries have continued to grow in diversity in terms of the supplements and additives offered as well as the types of consumers being targeted. One of the challenges faced by these industries is how to package and distribute supplements, pharmaceuticals and additives so that they can be easily carried and consumed at a convenient time by the consumer.

There is a pervasive trend in the supplement industry, and particularly in the protein powder industry, to sell the supplement in a loose powder form packaged inside of a tub with a scooper. The scooper is generally kept inside the tub, and in order to consume the powder a consumer will generally open the tub, scoop out the desired amount of supplement powder into a container, and then mix the supplement powder with water until it forms a suspension and can be consumed. This process is not only time consuming, but requires that the user know the appropriate amount of protein that needs to be consumed. The biggest problem with the current packaging and distribution process is that the tubs commonly used to store and sell the supplement powder are not convenient for carrying around, e.g., to take to the gym or carry with you to work, etc. Moreover, such delivery mechanisms do not allow for easy insertion into the necks of plastic water bottles and other beverages.

In addition, it is unhygienic for a consumer to reach a scooper into the tub with uncovered hands, especially if the consumer is sweaty after a workout. There is also a lack of precision and control over the quantity of protein included in each shake due to the fact that the scooper is of a uniform size and there is no convenient or accurate way to measure out a fraction of a scoop. If multiple supplements are to be included in a shake, the process of opening the tub, measuring the powder, pouring it into the container, replacing the scooper, and closing the tub must be performed for each supplement.

Additionally, a need exists for the efficient delivery of additives such as cannabis derivatives. Existing delivery mechanisms, including edible food-based delivery systems and smoking mechanisms remain inefficient and associated with undesirable side effects. Such delivery mechanisms also are accompanied by undesirable pollutants. Cannabis derivatives and cannaboids often are mis-measured and mis-dosed. These undesirable aspects of existing cannabinoid delivery mechanisms remain particularly problematic in association with the delivery of cannaboids in association with medical usage. This is particularly the case with regard to THC-A, which has been found to have substantial benefit to the peripheral nervous system (for pain management), the gut and the immune system. A need exists for an improved delivery system that better delivers the more easily extracted THC-A, which is water-soluble and does not require heat to extract, and therefore more easily retains other beneficial compounds such as terpenes and CBD. Aspects of the present invention are directed to these and other problems.

### BRIEF DESCRIPTION OF THE DRAWINGS

The above and other aspects, features and advantages of certain embodiments of the present invention will be more apparent from the following detailed description taken in conjunction with the accompanying drawings, in which:

FIG. 1 illustrates a front plan view of a supplement tablet and packaging according to the present disclosure, with the tablet being expelled from the package into a container with liquid.

FIG. 2 illustrates a front plan view of the supplement tablet and packaging of FIG. 1 with several packages in a sheet.

FIG. 3 illustrates a bottom plan view of the supplement tablet and packaging of FIG. 1 having a removable portion.

FIG. 4 is a block diagram of a system for customizing the supplement tablet and packaging of FIG. 1.

While the invention is described with reference to the above drawings, the drawings are intended to be illustrative, and the invention contemplates other embodiments within the spirit of the invention.

### DETAILED DESCRIPTION

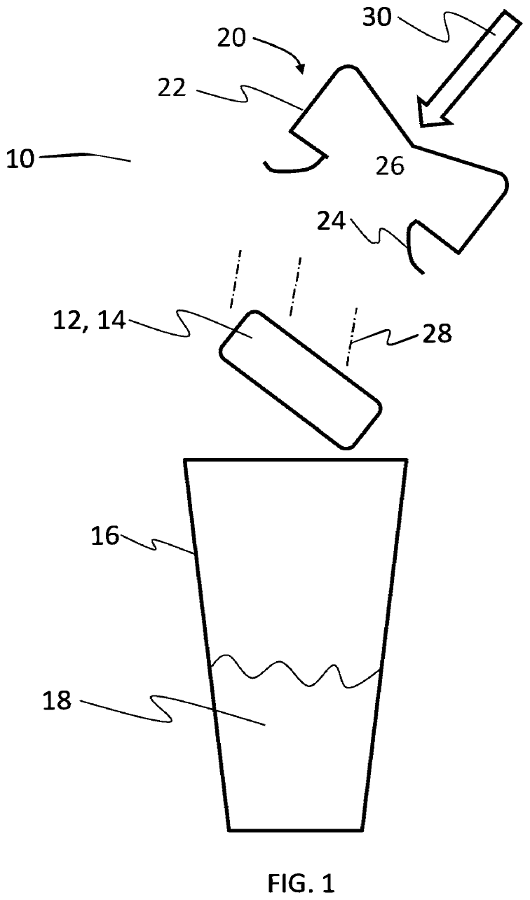

Referring to FIG. 1, the present disclosure describes a single use packaged product 10 including a tablet 12 containing supplement powder 14. In an embodiment, the tablet 12 is substantially round and of a diameter such that it can fit within a standard sized plastic water bottle having an opening of a diameter no greater than two inches. In an embodiment, the diameter of the tablet 12 is approximately 1 inch. The density of the supplement powder 14 is such that the tablet 12 will break apart to form a viscous consumable fluid, such as a shake (i.e., a “protein shake”, as in the drinks commonly consumed by people before/after weight training, and which commonly contain ingredients other than just protein; or a “cannabis shake”) when submerged and manually shaken in a container 16 of liquid 18. The supplement powder 14 in an embodiment of the invention includes a protein. Particularly in embodiments where the tablet 12 comprises supplement powder 14 that includes a protein, the tablet 12 is much larger, having a diameter of up to 3 inches in size. In alternative embodiments, particularly in embodiments wherein the supplement powder 14 includes a protein, the size of the tablet 12 is even larger, up to 6 inches in diameter and up to 3 inches in height. In an alternative embodiment, the supplement powder 14 in an embodiment of the invention includes a cannabis product. In an embodiment of the invention, the cannabis product comprises THC. In an embodiment of the invention, the cannabis product comprises THC-A. In various embodiments, the tablet 12 incorporates a matrix, which as one skilled in the art recognizes is the middle section or core of the tablet 12. In an embodiment, the matrix is comprised of the supplement powder 14.

Still referring to FIG. 1, the packaged product 10 also includes a package 20 having a first portion 22 and a second portion 24, which together defining a receiving area 26. The tablet 12 is initially disposed inside the receiving area 26, and enclosed by the package 20 for protection from contamination and accidental exposure to moisture. The package 20 is configured to expel the tablet 12 as indicated by motion lines 28, via manual manipulation of the package 20 as indicated by arrow 30.

In some embodiments, the manual manipulation is provided by a person depressing the center of the package 20 with their finger in order to push the tablet against the second portion and thereby cause it to break, in order for the tablet to be released from the package 20 and fall into the container 16 holding the liquid 18. The rounded side of the first portion 22 is structurally rigid so that the package 20 can withstand a compressive force sandwiching the entire package 20 without deformation of the package 20, thereby protecting the tablet 12 from being crushed. The flat side of the first portion 22, which is structurally flexible so that it can be pushed in to expel the tablet 12 with a relatively small force directed toward a central area of the flat side of the first portion 22. The flexible material is used so that the force (e.g. as indicated by arrow 30) on the central area of the flat side is substantially transferred to the tablet 12, and thereby substantially transferred to an interior side of the second portion 24. This causes the second portion 24 to break, releasing the tablet 12 from the package 20. The second portion 24 is a sheet of material that is malleable and has intentionally low shear strength, e.g. aluminum foil. The first portion is made from a material that is somewhat flexible and also generally water-resistant, e.g., a polymer.

In other embodiments, the tablet 12 has a coating on its outer surface. The coating adds dimensional stability and aids in the mixing of the tablet 12 with the liquid 18. The coating contains protein, and may also contain protein, vitamins, medicines, THC, THC-A, caffeine, and other agents or supplements that may be found in the supplement powder 14. In an embodiment, the coating serves as a moisture barrier. The present inventors recognize that to maintain optimal effervescent qualities, the matrix of the tablet should maintain optimized moisture levels in accordance with the teachings contained within U.S. Pat. No. 6,495,163 with a filing date of Sep. 23, 1998 which is hereby incorporated by reference in its entirety. The coating may be an effervescent coating to aid in the mixing of the tablet 12 with the liquid 18, or the tablet 12 itself may be an effervescent tablet due the incorporation of effervescent substances within its composition. In such embodiments, the effervescence may be accomplished through the incorporation of sodium bicarbonate, and/or potassium bicarbonate in the composition of supplement powder 14 and/or in the coating of the tablet. In an alternative embodiment, the effervescence may be accomplished through use of arginine in the supplement powder 14 and/or in the coating of the tablet. The present inventors have recognized the benefit of using arginine as a non-sodium alternative to provide effervescence, as many seek to avoid the addition of sodium to their diets. These ingredients react with water to create an acid base reaction releasing CO₂ and aiding in the breakup of the tablet 12. As used herein, effervescent means that the substance (in either the coating and/or supplement powder 14) reacts with a liquid causing the release of gas. Moreover, in an embodiment, the tablet 12 may incorporate one or more disintegrants. Disintegrants in the context of the invention are described as substances that aid in the breakdown of the tablet 12 into a fluid. More precisely, disintegrants are described as substances intended to aid in the break up of the compacted mass of the tablet 12 when it is put into a fluid environment. Disintegrants promote moisture penetration and dispersion of the tablet matrix. In embodiments of the invention, particularly embodiments that incorporate protein in the supplement powder 14, the tablet 12 incorporates Chitosan as a disintegrant. The present inventors have recognized that Chitosan assists with the breakdown of the tablet 12 containing a supplement powder 14 comprising protein once it reaches a wet substance. In alternative embodiments, the tablet 14 may incorporate any of the following as a disintegrant: crosscarmellose sodium, Ac-Di-Sol, primellose, vivasol, crosdpovidone, kollidon, polyplasdone, sodium starch glycolate, alginic acid, satialgine, soy, polysaccharides, emcosoy, and/or calcium silicate. In an embodiment, the inclusion of one or more disintegrants causes rapid dispersion of the tablet 12 once placed in a liquid. In an embodiment of the invention, the breakup of the tablet within the fluid would take approximately 15-20 seconds to fully disperse within the fluid if the supplement powder 14 contains protein. In an embodiment where the supplement powder 14 contains a cannabis derivative, the time to dispersion would take approximately 10-15 seconds. While effervescent typically refers to the release of carbon dioxide, it can refer to other gases escaping as well. In embodiments of the invention, the supplement powder is configured to comprise a dosage of THC in an amount between 1 milligram and 10 milligrams. In embodiments of the invention, the supplement powder is configured to comprise an amount between 0.5 milligrams of CBD and 8 milligrams of CBD. In an embodiment of the invention, the supplement powder is configured to contain precisely 5 milligrams of THC and 2 milligrams of CBD. The present inventors have recognized that THC-A provides many of the desired physiological effects of THC, however, due to being acidic and water-soluble, THC-A does not cross the blood brain barrier and therefore does not create undesirable psychological effects. In varying embodiments of the invention, therefore, within the supplement powder, THC-A is used in place of THC.

In yet other embodiments, there is a second tablet 12 included in the receiving area 26 that does not have protein in it. In yet alternative embodiments, there is a second tablet 12 included in the receiving area 26 that does not have cannabis, a cannabinoid, THC, or THC-A in it. The second tablet 12 would have one or more alternative supplements selected by the consumer. In other embodiments, a third tablet 12 (or multiple additional tablets 12) may be included in the receiving area 26. For embodiments having more than one tablet 12, an advantage is provided in that the consumer can see each supplement that they are taking in a separate tablet form, while still being able to store and consume them all at once. In other embodiments, multiple tablets 12 are used to aid in the speed of mixing the tablet 12 with the liquid 18, and the multiple tablets 12 do not necessarily have a different composition.

In other embodiments, the shake formed by mixing the tablet 12 with the liquid 18 is a suspension where the supplement powder 14 is suspended in water. In yet other embodiments, the supplement powder 14 forms a solution when mixed with water. The tablet 12 is designed to break apart in water relatively quickly, for example after being manually shaken in a container 16 of liquid 18 for a period of time in the range of 1 second to 60 seconds.

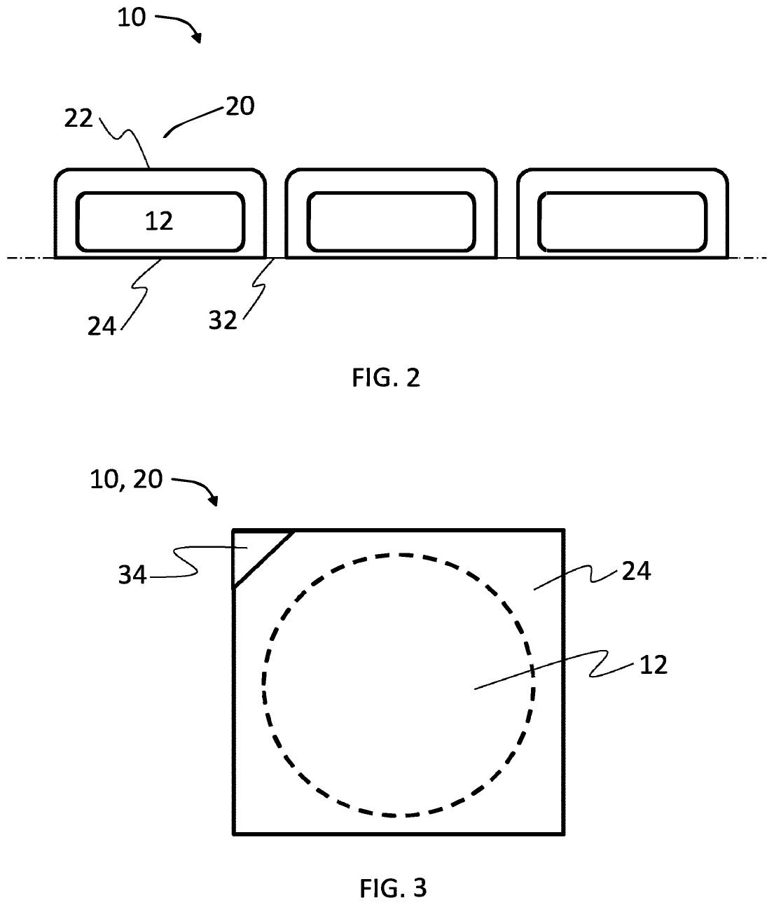

Referring to FIG. 2, the packaged product 10 is shown with the tablet 12 contained in the package 20. The first portion 22 of the package 20 is attached to the second portion 24. A sheet 32 connects the package 20 to other packages 20 such that multiple packages 20 can be transported easily. In other embodiments, the sheet 32 connects the first portion 22 of the package 20 to the first portion 22 of a neighboring package 20. The sheet 32 may be designed such that the packages 20 are all aligned in one row, or it may be designed such that the packages are in a 2×2 square (i.e., two packages 20 across and two packages 20 down), or 2×3, or 2×4 rectangle, or other configurations known by those of skill in the art familiar with the present disclosure. In other embodiments, the package 20 is a stand-alone item which is distributed with other packages 20 in a box or a tub, and is not connected to the other packages 20 in the box.

In some embodiments, the receiving area 26 is shaped to receive the tablet 12 such that a ratio of a receiving area height to a receiving area width is approximately equal to a ratio of a tablet height to a tablet width. The package 20 is cylindrically shaped, and the rounded side of the first portion 22 and the first flat side of first portion 22 defines the receiving area 26, and the second portion 24 is a removable portion or a breakable portion.

Referring to FIG. 3, the packaged product 10 is shown with the dotted line indicating the position of the tablet 12 behind the second portion 24 of the package 20. The second portion 24 has a tab 34 which is designed to help with the removal of the second portion 24 from the first portion 22 in order to expose the tablet 12 just prior to removal from inside the package 20. The tab 34 is designed as a corner of the second portion 24 that is not adhesively connected to the first portion 22, thus allowing a person to grip the tab 34 and peel the second portion 24 off of the first portion 22. This design for opening the package via manual manipulation is an alternative to the method described above (and as shown in FIG. 1.) where the tablet 12 is “popped out” of the package 20 via a break in the second portion 24. In other embodiments, the package 20 is torn open, with one or both of the first portion 22 and second portion 24 being tearable. In yet other embodiments, the first portion 22 has a form fit with the second portion 24 and the two parts can be popped apart to release the tablet 12. Other variations of opening the package 20 are known by those of skill in the art having the benefit of the present disclosure.

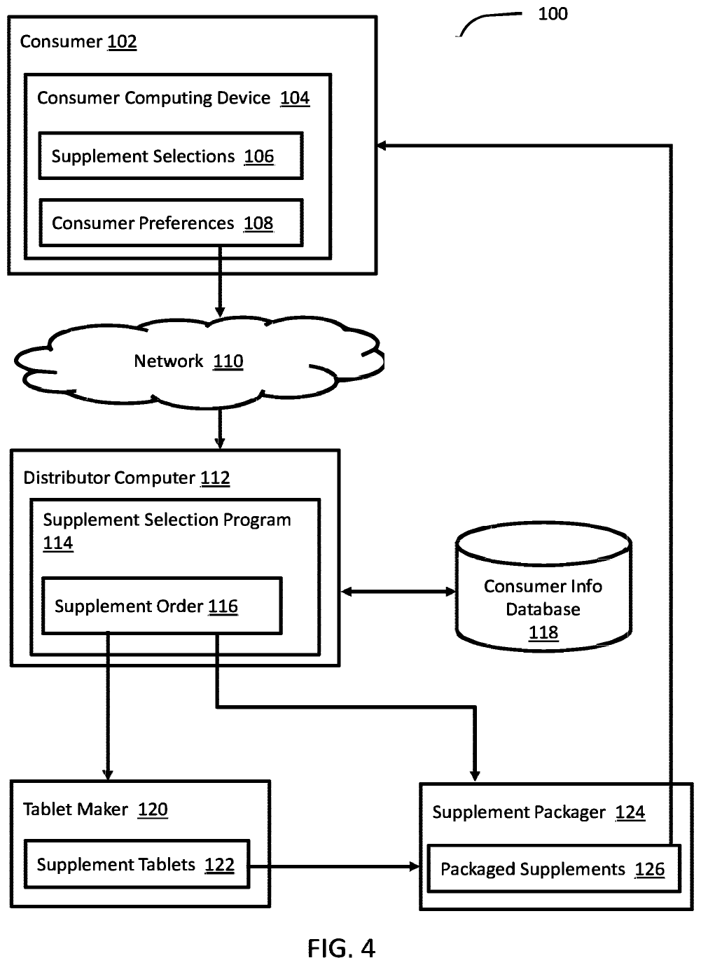

Referring to FIG. 4, a block diagram of a system 100 for customizing the packaged product 10 is shown. The system 100 includes a consumer 102, which operates a consumer computing device 104 to input supplement selections 106 and consumer preferences 108. The supplement selections 106 and consumer preferences 108 are sent to a distributor computer 112 through a network 110. The distributor computer 112 has a supplement selection program 114 executing thereon for generating a supplement order 116 using the information provided in the supplement selections 106 and the consumer preferences 108. The distributor computer 112 may store information such as the supplement selections 106, consumer preferences, or supplement order 116 on a consumer information database 118 for later usage.

Still referring to FIG. 4, a tablet maker 120 receives the supplement order 116 and uses it to form supplement tablets 122 having the right supplements, e.g., supplement type, quantity, flavor, etc. The supplement tablets 122 are compressed by the tablet maker 120 to an appropriate density so that they will break apart when shaken in water, but not break apart during typical handling and transport. The supplement tablets are received by the supplement packager 124 which packages the tablets 122 to form packaged supplements 126, which are then sent to the consumer 102.

In some embodiments, the supplement selections 106 may include information such as protein type, protein flavor, cannabis type, cannabis flavor, protein quantity, cannabis quantity, THC quantity, THC-A quantity, vitamins, minerals, and nutrients that a consumer 102 wishes to be included in the packaged supplements 126 that are sent to them. In other embodiments, the consumer preferences 108 include information about the consumer's age, weight, sex, build, training goals, medical needs, and dietary restrictions, and the supplement selection program 114 determines the supplement selections 106 for a consumer 102 based on the consumer preferences 108 entered by the consumer 102. This embodiment allows the consumer 102 to receive a product tailored to their needs even if they do not have the expertise to make the selections on their own. In other embodiments, the consumer 102 can include their prescribed pharmaceutical drugs, allergy medication, daily vitamins, medical or recreational marijuana, and other substances with their supplement selections 106 so that they are given an all-inclusive tablet that meets their dietary needs. An advantage of this embodiment is that certain substances may be absorbed by the body faster when consumed in the form of a liquid solution or suspension.

The packaged product 10 offers several advantages over known products. Among other things, the packaged product 10 is designed for a single use, allowing a consumer to easily transport and consume their protein powder in the form of a tablet so that when the consumer wishes to make a protein shake, all they have to do is expel the tablet 12 from the package 20 and shake it up with water to create the shake. This allows, for instance, gym-goers to avoid having to use a scooper with a tub of protein which is bulky and inconvenient to carry around, and also requires measuring, which can be inconvenient and unhygienic. The package 20 also provides an advantage by being sized to fit in a clothing pocket or a purse, e.g. the pocket of athletic wear such as a small thigh pocket on running spandex designed for a key (i.e., a “key pocket”) or small zipper pocket in a fleece for storing a credit card (i.e., a “card pocket”). In other examples, the package 20 offers the advantage of being sized for concealment.

The packaged product system 100 also allows for increased efficiency since a consumer may customize their single use packaged product so that each tablet 12 has precisely the right supplements for the dietary needs. For example, an athlete taking multiple supplements seeking to control their supplement intake with precision and ease can benefit greatly from the packaged product system 100 since it replaces a series of complicated and time-consuming measurements of various powders and pills from multiple containers each time the athlete wishes to consume a supplement shake. Such consumption might also take place by anyone, athlete or otherwise, who wishes to improve their overall wellness. An athlete just starting out with a supplement regiment can also benefit from the packaged product system since it can aid a consumer in selecting the supplements they wish to take, or even calculate certain things for them, e.g., determine quantity based on their training goals/weight/sex, or e.g., determine protein type (plant or casein) based on dietary restrictions or allergies.

The packaged product 10 also provides an advantage since it may be used to deliver other ingestible items or combined with a user's other vitamins/medicine to meet the user's supplement/medical needs with one item. Advantages also include that large doses of ingredients can be taken in a single serving, and nutrients can be absorbed by the body faster than if taken in other forms such as pills.

With conventional solid tablets or capsules that are swallowed by a user, the tablet is transported to the user's stomach after being swallowed, where it dissolves gradually. The passage time varies in different people, depending on anatomical and physiological factors. In case of a longer passage time, solid dosage forms can dissolve partially and this may cause irritation of mucous membranes. Advantages of the packaged product 10 include that because it is dissolved in liquid the ingredients are evenly distributed in the solution, and high, localized concentrations cannot occur.

The present invention is a bulk powder, single use packaged effervescent tablet, or single use packet or sachet containing an effervescent supplement powder with a density that enables the effervescent tablet or effervescent powder to break apart and/or dissolve when it is added to or submerged in any liquid, including non-carbonated and carbonated liquids, with or without stirring. A carbonated liquid should induce more rapid carbonation and dissolution of the active ingredients (cannabinoids, terpenes and cannabidiol [CBD]) due to the inherent CO₂ levels of the carbonated liquid. Accordingly, the effervescent components in the supplement powder and the floating delivery system allow for convenient administration in non-carbonated and carbonated liquids. The phrase “supplement powder” as used herein refers to a powder comprising a variety of additives including diluents (e.g., mannitol, sorbitol, xylitol, pearlitol), emulsifiers (e.g., lecithin, locust bean gum, xanthan gum), sweeteners (e.g., sucralose, isomalt, maltitol, aspartame), and lubricants (e.g., sodium benzoate, polyethylene glycol, adipic acid) which facilitate the dispersion of the active ingredients hemp or cannabis extract to include all cannabinoids and terpenes, including cannabidiol (CBD) into a liquid.

The invention is for the use as a bulk powder supplement, tea mixture, aquaculture, or pet formulation:

Potassium Bicarbonate (100-400 mg)

Citric Acid (100-200 mg)

Pearlitol (10-40 mg)

Isomalt (10-20 mg)

Lecithin (0.100-0.400 mg)

Adipic acid (1-5 mg)

Cannabinoids, terpenes, and cannabidiol [CBD] (1-20 mg)

The active ingredients in the present invention are subjected to a micro jet mill process that yields ingredients of 1.5-2 micron size. A mixture of several ingredients is then oscillated together in a Tubular mixer in order to achieve an evenly distributed blend. Certain ingredients (preservatives) in the bulk powder are slightly larger in particle size, and micro jet mill processing creates a consistent and uniform powder blend of an optimal micron size. The micro jet mill process and use of Tubular mixer yield specific micron sizes for each ingredient in the effervescent bulk powder. When this method is applied to the proprietary bulk powder blend, a water soluble effervescent powder is achieved. Effervescent tablets and powders are produced in controlled environmental conditions through various methods that include: direct compression, wet granulation, fluid-bed granulation, and fusion method. As the proposed invention is sensitive to temperature and moisture, modest temperatures (e.g., 25° C.) and a relative humidity of approximately 25% or less must be held constant during production to prevent granulation or adhesion of the effervescent tablet powder to the machinery due to moisture absorption.

The effervescent powder mix itself contains emulsifiers that aid in the mixing of the bulk powder with the liquid and promotes dispersion when the supplement is added to a liquid. The present inventors recognize that in order to maintain optimal effervescent qualities, the matrix of the effervescent powder should maintain optimized moisture levels in accordance with the U.S. Pat. No. 6,495,163 with a filing date of Sep. 23, 1998 which is hereby incorporated by reference in its entirety.

For this invention, the effervescence has been accomplished through the incorporation of sodium or potassium bicarbonate or carbonate (or any other carbonate/bicarbonate consisting of alkali metals) and citric acid (or tartaric acid, malic acid, or any other acid anhydride) in the composition of the effervescent tablet or effervescent powder and/or in the coating. These ingredients react with water to create an acid-base reaction that releases CO₂ and aids in dispersion of the effervescent tablet or effervescent powder. The CO₂ that is produced through an effervescence reaction increases the penetration of the active substances (hemp or cannabis extract to include CBD) into the paracellular pathway and subsequently increases ingredient absorption. As used herein, effervescent means that the substance in the supplement powder reacts with a liquid and causes the release of gas.

Moreover, the effervescent powder may incorporate diluents in the context of the invention. Diluents are described as substances that aid in the breakdown of the bulk powder into a fluid. More specifically, diluents are described as substances intended to aid in the breakup of the compacted mass of the tablet when it is put into a fluid environment. Diluents promote moisture penetration and dispersion of the powder. Naturally-sourced diluents include starches, partially pre-gelatinized starches, and hydrolyzed starches. Common diluents for supplement formulation include lactose monohydrate and anhydrous lactose as well as sugar alcohols such as mannitol, sorbitol, pearlitol, and xylitol. Accordingly, as the invention incorporates hemp or cannabis extract to include CBD, all cannabinoids and terpenes in the supplement powder, the effervescent powder incorporates one or multiple diluents that assist the breakdown of the tablet once it reaches a liquid. In addition, the invention incorporates one or multiple emulsifiers that promote the dispersion the active ingredient hemp or cannabis extracts, cannabinoids including CBD and/or terpenes (oil-based substances) in liquid. Common emulsifiers include lecithin, locust bean gum, xanthan gum, sodium mono- and di-glycerols, ammonium phosphatide, and stearoyl lactylate. The invention also incorporates one or multiple sweeteners that are hygroscopic in nature and increase the bulk of the effervescent tablet or powder. Common sweeteners include sucralose, maltitol, isomalt, and aspartame. Furthermore, one embodiment of the invention incorporates one or multiple water soluble lubricants (adipic acid, sodium benzoate, and polyethylene glycol) to prevent the adhesion of the effervescent tablet or powder to the device and restrict the formation of insoluble scum on the liquid surface. Additional aspects of the invention are tailored to allow for optimal dissolution time in hot and cold liquids, in different types of liquids, and in the dissolution and delivery of multiple doses.

The dispersion of the effervescent powder within the fluid may take approximately 5-20 seconds or longer to fully disperse within the liquid if the supplement powder contains hemp or cannabis extracts, cannabinoids including CBD and/or terpenes. The effervescent powder is designed to break apart and/or dissolve in 3 to 4 ounces of water relatively quickly, for example, after being added or stirred in a container of liquid for a period of time in the range of 1 second to 60 seconds. Effervescent generally refers to the release of CO₂, but it can also refer to other gases escaping. In the present invention, the supplement powder is configured to comprise an amount of hemp or cannabis extracts, cannabinoids including CBD and/or terpenes between 1 milligram and 200 milligrams per dosage. The main advantage of the effervescent configuration is that the active ingredients (e.g., CBD) may be absorbed by the body faster when they are consumed in the form of a liquid solution or suspension.

That is, effervescent delivery systems are low-density formulations that stay afloat over the gastric fluid and remain buoyant in the stomach without adversely affecting the rate of gastric emptying. This system yields better absorption and increased bioavailability. The buoyancy of the delivery system is assisted by low density agents and compounds such as sodium bicarbonate, citric acid, tartaric acid, or other metal carbonates or alkali metal bicarbonates. These agents present in solid (powder) form and are capable of releasing CO₂ when they are formulated with active ingredients and other auxiliaries to provide granules without the premature evolution of CO₂. The delivery system utilizes effervescent reactions when the bulk powder comes in contact with the gastric fluid due to the CO₂ gas that is generated from the system when it is exposed to liquid as water or other liquids act as catalyzing agents that increase the rate of the reaction. The concept of effervescent powders is mainly based on the mixture of ingredients such that the active ingredients remain embedded in the delivery system in which the active ingredients are released without the disintegration of the bulk powder. The effervescent powder may also be used as a sustain release dosage form to overcome problems that are associated with conventional dosage forms. This also reduces fluctuations of active ingredient(s) concentration and enhances the bioavailability of the bulk powder.

The floating delivery system is designed to yield an effervescent powder bulk density that is lower than that of gastric fluid in order for the active ingredients to remain buoyant for extended time periods without affecting the gastric system's emptying rate. The floating delivery systems can be categorized as a non-effervescent system or an effervescent system. In a non-effervescent floating delivery system, the active ingredients swell in the gastric fluid while maintaining relative stability of their shape and a bulk density that is less than the density of the gastric fluid. This augments the floating process of the active ingredient serving forms.

The present invention incorporates an effervescent floating delivery system that is based on effervescent components (e.g., potassium bicarbonate, citric acid) which will liberate CO₂ due to the acidic properties of the gastric fluid. The liberated CO₂ gas will become entrapped in the floating layer formed by the hydrocolloids (e.g., lecithin, locust bean gum, xanthan gum) and this reaction leads to an upward motion of the active ingredients that also maintains its buoyancy.

The floating delivery system of effervescent formulations might offer faster biological effects than non-effervescent formulations. A non-effervescent solution may also have a bad taste and high viscosity that might increase its intolerability in the digestive tract. Furthermore, an effervescent formulation increases the ease of administration and improves the absorption of the active ingredients through the previous dissolution in a buffered liquid medium. In addition, an effervescent delivery system can buffer the aqueous solution of the active ingredients, thereby increasing the stomach pH which prevents the inactivation or degradation of the active ingredients. The buffering effect through carbonation may also induce quicker emptying of the stomach, usually within 20-30 minutes into the small intestine as this promotes maximum absorption of active ingredients.

Accordingly, effervescent formulations are advantageous as the active ingredients (cannabinoids, terpenes and cannabidiol [CBD]) are already in solution at the time they are consumed, which makes the absorption faster and more complete in comparison to non-effervescent formulations. The floating delivery system enables the ingredients to fully dissolve in a buffered solution and this reduces localized contact in upper gastrointestinal tract. This results in less irritation and greater tolerability of the solution. This buffering mechanism also prevents gastric fluids from interacting with active ingredients themselves, which is a major causative factor for esophageal and intestinal upset. Moreover, this system produces a homogenous solution that may have better consumer appeal than viscous, non-effervescent solutions. Additionally this delivery system helps the active ingredients retain their palatability after lengthy storage times. Excellent stability is also inherent to an effervescent formulation, particularly surpassing that of non-homogenous liquid solutions.

Active ingredients delivered through effervescent delivery systems also have reproducible and predictable bio-kinetic profiles that are more consistent than capsules or tablets as effervescent components assist the therapeutic profiles of the active ingredients. Effervescent components also facilitate the solubilization of ingredients that generally demonstrate poorly solubility (cannabinoids, terpenes and cannabidiol [CBD]). An effervescent delivery system enhances the penetration of a broad range of active ingredients that vary in size, structure, and other physiological properties. In addition, the effervescent formulation can be used for programmed product administration in remote areas and this type of formulation addresses the issue of physicochemical stability and the high cost of transporting capsules, tablets, or viscous syrups. Therefore, an effervescent tablet or powder provides a realistic solution to easy administration, maximum absorption, sustained active ingredient stability in the gastrointestinal tract, and a low incidence of stomach upset, in addition to other advantages.

Accordingly, the packaged effervescent product offers several additional advantages over known products. For example, the packaged product is designed for single use, allowing a consumer to easily transport and consume the hemp or cannabis derivatives in the form of a tablet so that when consumers wish to make a drink, all they have to do is expel the tablet from the package and add it to or shake it up with water or another liquid to create the drink. This allows consumers to avoid having to use a scooper with a tub of supplement powder that is bulky and inconvenient to carry around, and also requires measuring, which is another factor that is inconvenient and unhygienic. The package also provides an advantage of being sized to fit in a clothing pocket, purse, or other areas that offer concealment. Advantages also include that large doses of ingredients can be taken in a single serving, and nutrients such as terpenes derived from CBD can be absorbed by the body faster than if they are taken in other forms such as pills.

In particular, the effervescent delivery of hemp extract, cannabis extract, CBD, other cannabinoids terpenes, flavonoids, and other agents (e.g., proteins, extracts), provide highly bioavailable active ingredients of substantial benefit to the peripheral nervous system (for pain management), the immune system, and the gut due to anti-inflammatory and antioxidant properties. Accordingly, an effervescent delivery system is an improved method that better delivers the oil-based and easily extracted active ingredient(s). Furthermore, cannabinoids including CBD, terpenes and other hemp and cannabis extracts do not require heat in order to be extracted and thusly effectively retains additional beneficial compounds (e.g., vitamins, minerals, flavonoids, terpenes, essential fatty acids). With conventional solid tablets or capsules that are swallowed by a consumer, the tablet is transported to the consumer's stomach after being swallowed, where it dissolves gradually. The passage time varies in different people, depending on anatomical and physiological factors. In cases of longer passage times, solid dosage forms can dissolve partially and this may cause irritation of mucous membranes. Advantages of the packaged effervescent product include that because it is dissolved in liquid, the ingredients are evenly distributed in the solution and high localized concentrations cannot occur. Furthermore, an effervescent powder that is dissolved prior to ingestion maximizes the absorption rate and bioavailability by ensuring that CBD is fully dissolved in liquid before it is delivered to its absorption sites, which are primarily the stomach and the intestine. Dissolution of active ingredients in liquid is a prerequisite to absorption into the systemic (blood and lymphatic) circulation. The series of steps that are required for stomach and intestinal absorption of tablets and capsules are bypassed by administering pre-dissolved (effervescent) products. Therefore, the active ingredient(s) is available earlier for absorption, and there is no guarantee that a solid supplement form will release all of the active ingredients contained within it before it transits through intestinal tract to the absorptive sites. Formulations in which the active ingredients are present in dissolved form before it reaches the absorptive sites usually exhibit greater bioavailability. Thusly, the present intervention demonstrates an optimal delivery system in comparison to current delivery systems for hemp and cannabis such as tablets, capsules and powders which are not very bioavailable.

While several embodiments have been disclosed, it will be apparent to those of ordinary skill in the art that aspects of the present invention include many more embodiments and implementations. Accordingly, aspects of the present invention are not to be restricted except in light of the attached claims and their equivalents. It will also be apparent to those of ordinary skill in the art that variations and modifications can be made without departing from the true scope of the present disclosure. For example, in some instances, one or more features disclosed in connection with one embodiment can be used alone or in combination with one or more features of one or more other embodiments.

What is claimed is:

1. An effervescent product, comprising:
an effervescent supplement powder with a density that enables the effervescent powder to break apart and/or dissolve when it is added to or submerged in any liquid, with or without stirring.

2. The effervescent product of claim 1, wherein the product is a bulk powder.

3. The effervescent product of claim 1, wherein the product is a single use tablet.

4. The effervescent product of claim 1, wherein the product is a single use packet.

5. The effervescent product of claim 1, wherein the product is a single use sachet.

6. The effervescent product of claim 1, wherein the product is a beverage.

7. The effervescent product of claim 1, wherein the effervescent supplement powder comprising a plurality of additives which facilitate the dispersion of active ingredients hemp or cannabis extract into a liquid.

8. The effervescent powder of claim 7 wherein the plurality of additives include diluents, emulsifiers, sweeteners, or lubricants.

9. The effervescent powder of claim 7 further comprising all cannabinoids, terpenes and cannabidiol (CBD) dispersed into a liquid.

10. A process for producing a water soluble effervescent powder comprising the steps of:
combining a plurality of ingredients wherein at least one ingredient is hemp or cannabis into a mixture;
introducing the mixture into a micro jet mill;
milling the mixture to form a powder blend;
introducing the powder blend into a mixer; and
mixing the powder blend to form a water soluble effervescent powder.

11. The process of claim 10 wherein the plurality of ingredients further comprising at least one of cannabinoids, terpenes or cannabidiol (CBD).

12. The process of claim 10 further comprising the step of:
adding a diluent.

13. The process of claim 10 further comprising the step of:
adding an emulsifier.

14. The process of claim 10 further comprising the step of:
adding a sweetener.

15. The process of claim 10 further comprising the step of:
adding a lubricant.

16. The process of claim 10 wherein the powder blend is 1.5-2.0 microns.

17. The process of claim 10 wherein water soluble effervescent powder contains between about 1-200 milligrams of hemp, cannabis, or cannabinoids per dosage.

18. A carbonated beverage, comprising:
an effervescent supplement powder with a density that enables the effervescent powder to break apart and/or dissolve when it is added to or submerged in any liquid, with or without stirring.

19. The carbonated beverage of claim 17, wherein the supplement powder includes hemp.

20. The carbonated beverage of claim 17, wherein the supplement powder includes cannabis.

21. The carbonated beverage of claim 17, wherein the supplement powder includes cannabinoids.
